(19) United States
(12) Patent Application Publication
(10) Pub. No.: US 2012/0046629 A1
(43) Pub. Date: Feb. 23, 2012

(54) Flow away garment

(76) Inventor: James Ronald Hannah, Jackson, MS (US)

(21) Appl. No.: 13/137,464

(22) Filed: Aug. 18, 2011

Related U.S. Application Data
(60) Provisional application No. 61/375,639, filed on Aug. 20, 2010.

(51) Int. Cl. A61F 13/49

(52) U.S. Cl. 604/385.01

(57) ABSTRACT

A blue pad liner designed for men, in a disposable diaper

This application claims benefits of a provisional application #61/375,639 Date Aug. 20, 2010

### BACKGROUND OF THE INVENTION

Chemicals in disposable diapers combine with urine can burn the skin. One of the caustic agents contained in urine is ammonia. Ammonia increases the ph level of the skin causing irritation. Ammonia is also used by bacteria as a source of nutrition, contributing to the reproduction of more micro-organisms. Without proper treatment, the cycle goes on and on/Being in contact with wetness over a period of time will cause skin break downs. Disposable diapers hold in urine against the skin. The dark damp and moist environment created by a diaper is a perfect breeding ground for bacteria cal infections. Wetness is one half the cause of diaper rashes, skin, and tissue break downs. After diaper change the effects of urine is on the skin. Cleaning up can be damaging with the rubbing, and the different types cleaning solutions can be abrasive to the skin. Irritations to the skin can be prevented, by frequent diaper changes but it would be costly.

### BRIEF SUMMARY OF THE INVENTION

The advantage of this disposable diaper is the flow away blue pad, designed for men. It has an elastic opening in the center for the penis to be inserted through. Once the penis is inserted it is protected by the back covering on the blue pad. This blue pad is made to shield the body from urine and the chemicals of the disposable diaper. It minimizes the clean up of urine on the skin. The Flow Away blue pad keeps the body from direct contact with the wetness of the disposable diaper's absorbent pad.

### DETAILED DESCRIPTION OF THE INVENTION

The Flow away pad which is a blue pad made in the shape of a liner for a disposable diaper with an elastic opening in the center for the penis to be inserted through. Once the penis is inserted it is protected by a covering on the back of the blue pad. It is a disposable diaper made with the blue pad built into it. The blue pad has a plastic covering on the back to keep the urine from coming in contact with the skin. All elements are necessary.

### FIGURES

FIG. 1. The elastic opening for the penis to be inserted.

FIG. 2. The blue pad diaper liner.

FIG. 3. The protective covering for the penis.

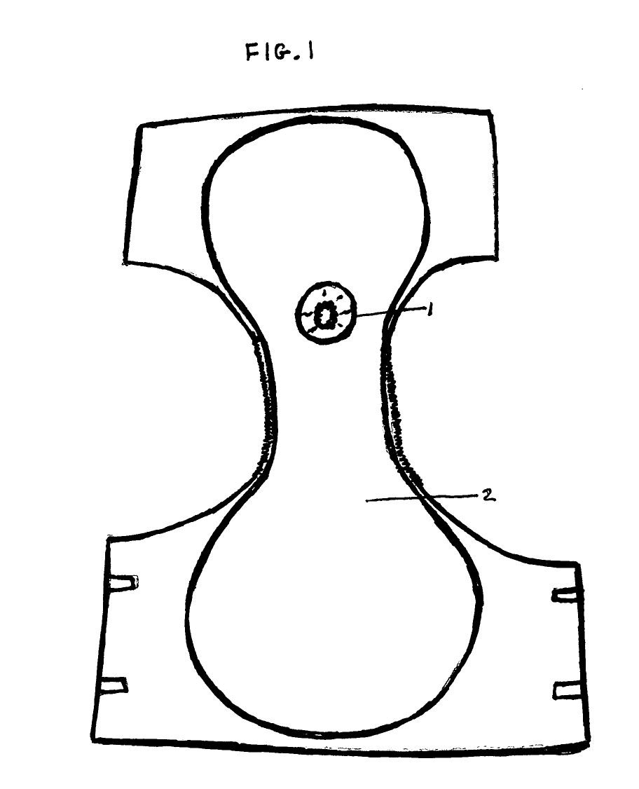

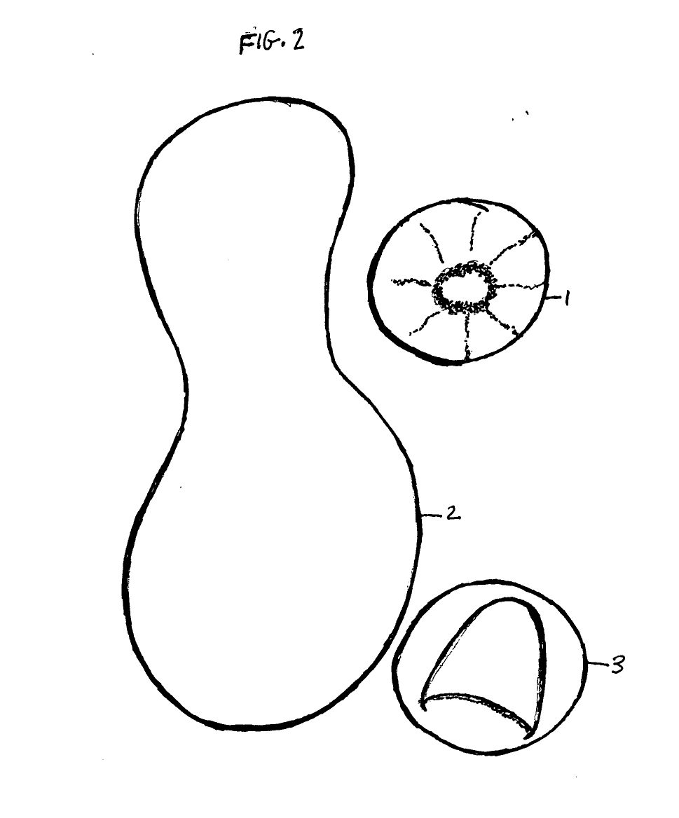

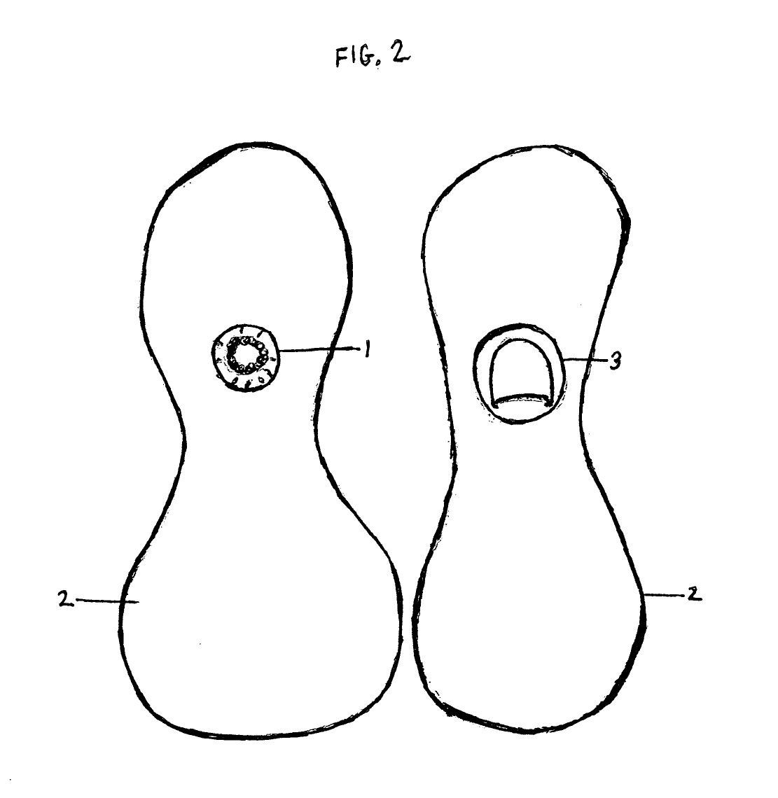

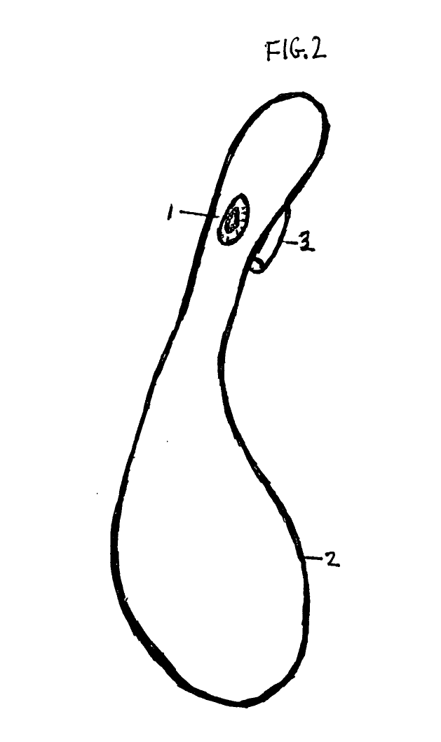

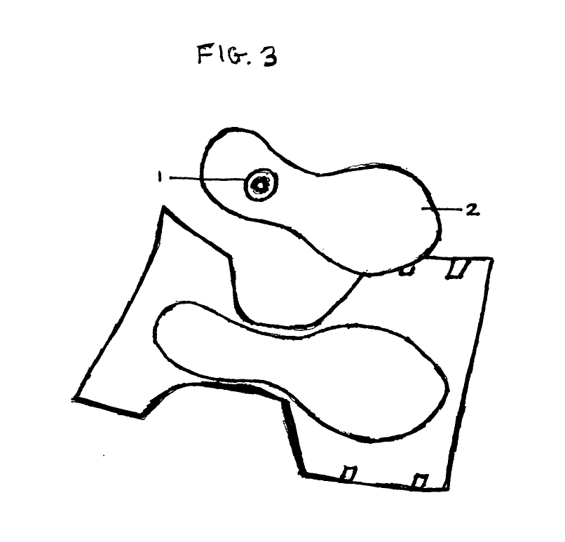

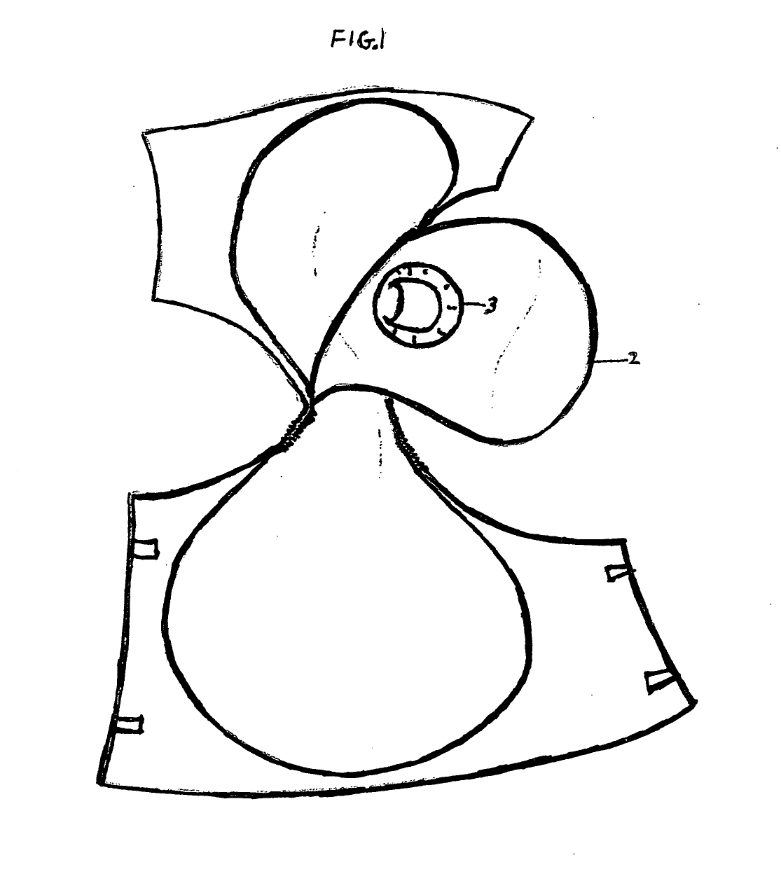

1. A blue pad built into the surface of a diaper to shield the body from urine, made for males.
